(19) United States
(12) Patent Application Publication
(10) Pub. No.: US 2019/0376942 A1
(43) Pub. Date: Dec. 12, 2019

(54) METHOD FOR DETERMINING CASEINS AND SERUM PROTEINS IN RAW OR LOW PASTEURISED DAIRY PRODUCTS

(71) Applicant: Spectralys Innovation, Romainville (FR)

(72) Inventor: Inès Birlouez-Aragon, Ermont (FR)

(21) Appl. No.: 16/436,309

(22) Filed: Jun. 10, 2019

(30) Foreign Application Priority Data
Jun. 8, 2018 (FR) 1855047

(51) Int. Cl. G01N 33/04; G01N 21/64

(52) U.S. Cl. CPC G01N 33/04; G01N 21/6486

(57) ABSTRACT

A method for determining a content of caseins and a content of serum proteins in a liquid foodstuff includes a) forming a solution of the liquid foodstuff by adding a buffer with a predetermined pH, the solution including the serum proteins and the caseins; b) stirring the solution to form a homogeneous mixture of the serum proteins and caseins; c) acquiring frontal fluorescence spectra of the mixture resulting from the excitation of the homogeneous mixture by at least one electromagnetic radiation with a determined wavelength; d) diluting the mixture by adding a predetermined quantity of water to separate by filtration the soluble serum proteins from the coagulated caseins suspended in the solution; e) acquiring right-angle fluorescence spectra of the diluted mixture, and f) determining the content of caseins and the content of serum proteins of the liquid foodstuff.

### CROSS REFERENCE TO RELATED APPLICATIONS

This application claims priority under 35 U.S.C. § 119(a) to French Patent Application Serial Number 1855047, filed Jun. 8, 2018, the entire teachings of which are incorporated herein by reference.

### BACKGROUND OF THE INVENTION

### Field of the Invention

The present invention relates to a method for evaluating the content of proteins of a foodstuff. In particular, the invention relates to a method for dosing simultaneously the caseins and the serum proteins contained in a liquid protein foodstuff, such as raw or low pasteurized milk.

### Description of the Related Art

In the context of milks or cheeses, two types of proteins are typically present: on the one hand, caseins, which are insoluble coagulable micelles under the effect of rennet, and on the other hand, serum proteins, soluble, contained in milk serum. In particular, raw or low heat-treated products contain serum proteins that are not denatured, for example under the effect of heat, and that can be precipitated, as well as the coagulable proteins that include these products.

With regard to this composition, it is known that the content of proteins and caseins present is fundamental to determine the technological quality of milk or a cheese including them, as well as the yield thereof, the texture thereof, and more generally the qualities of the finished product.

In this context, cheese manufacturers and manufacturers in the dairy sector must control content of casein fluctuations of milk, as these directly affect the cheese yield. For example, to guarantee an optimal quality, the cheesemaker should preferably maintain a constant rate of proteins and a constant cheese weight in each product. It is therefore necessary to anticipate the volume of product to be treated to obtain a quantity of caseins, therefore of curd, equivalent or even equal.

Thus, to maintain a constant volume of curd and to meet regulatory standards, manufacturers have to correct, in retrospect, the weight of the curd. It is much more cost-effective to adapt, beforehand, the milk volume implemented according to the casein dosage, the knowledge of which ensuring the conformity of the finished product.

Moreover, the dosage of serum proteins makes it possible to establish the ratio between these proteins and the caseins in a foodstuff, this ratio being defined as a mathematical division of the content of serum proteins by the content of caseins. However, this ratio is important to control for the manufacture of products such as yoghurts or infant formulas, as it influences the firmness and texture thereof, or the digestibility thereof, respectively. Moreover, the ratio between serum proteins and caseins is a regulated criterion for the labelling of infant formulas, and must be accurately reported. This ratio also conditions the cheese yield if the cheese milk is thermally treated, due to precipitation of the denatured proteins with the caseins that are present. Knowledge of this ratio thus makes it possible to target the pasteurization temperature of the product, to control the proportion of serum proteins that will be denatured, and therefore, to target the desired values of the ratio of denatured serum proteins on caseins.

To date, a known and standardized method, the Kjeldahl method, for dosing caseins and serum proteins includes the use of apparatuses and solvents, which requires laboratory practice. The result of the analysis is therefore delayed with respect to the collection of the sample from the foodstuff, and does not make it possible to adapt the treatment processes of this in good time.

Another known method aims to dose the total proteins by infrared and/or near infrared spectroscopic analysis. Implementing a complex calibration to be frequently adapted, this method enables the dosage of caseins in milk. However, this method does not make it possible for the dosage of denatured serum proteins, which are of nutritional interest. By performing the mathematical subtraction between the total content of proteins and the content of caseins, these two contents being measured by infrared and/or near infrared spectroscopy, the sum of the soluble content of serum proteins and of the proteose-peptone fractions is obtained, non-denatured, and without known interaction with the caseins.

In particular, it is known that the use of fluorescence spectrometry makes it possible to measure the serum proteins of a sample, for example a sample of raw milk, by precipitation of the caseins using an adequate buffer. By means of a spectrometer, it is then possible to measure the fluorescence of the marking amino acids of the protein concentration, and in particular the fluorescence of tryptophan, component of the denaturable serum proteins, within the sample

Moreover, methods are known aiming to measure the total content of proteins in samples by fluorescence after having rendered them transparent. These methods make it possible to calculate the casein rate by subtraction between the total protein rate and the soluble protein rate.

Examples of methods relying on the use of a right-angle fluorescence measurement in a transparent medium to determine the protein and/or casein content of a sample consists of precipitating a milk sample by means of an acid buffer. After around 5 minutes, a right-angle fluorescence measurement makes it possible to deduce the total content of proteins with a degree of error of around 3%. Moreover, another method would be to render transparent a milk sample by means of a buffer of impact ionic strength, to destabilise the micelles of caseins and of suitable pH. After around 3 minutes, a measurement of right-angle fluorescence is then performed to deduce the total content of proteins with a degree of error of around 5%. On the basis of the same milk sample, the subtraction of the content values thus measured of the total proteins and of soluble proteins makes it possible to deduce the content of caseins.

However, the Kjeldahl method is complex, polluting and costly, and the latter method is relatively cumbersome to implement in an industrial context. In particular, the latter method according to the prior art has three major disadvantages. Firstly, it is slow, since around 8 minutes are needed to measure the casein rate. Secondly, the subtraction of the total content of proteins and the soluble content of proteins features a significant degree of inaccuracy, of around 8%, by cumulative addition of the errors. Thirdly, these methods are relatively not easy to use, since two sample preparations are necessary.

### BRIEF SUMMARY OF THE INVENTION

In order to overcome at least one of the abovementioned disadvantages, one aim of the present invention is to propose a method for simultaneously dosing the caseins and serum proteins that includes a liquid protein foodstuff, such as raw or low pasteurised milk.

For this purpose, an aim of the invention relates to a method for determining a content of caseins and a content of serum proteins in a liquid foodstuff such as raw or low pasteurised milk, characterised in that the method includes successive steps consisting of:
forming a solution of the liquid foodstuff by adding a buffer with a predetermined pH, the solution including the serum proteins and the caseins, the caseins being in a coagulated form suspended in the solution and the serum proteins are solubilised in the solution;
stirring the solution to form a homogeneous mixture of the serum proteins and caseins;
acquiring a plurality of frontal fluorescence spectra of the mixture, the frontal fluorescence spectra resulting from the excitation of the homogeneous mixture by at least one electromagnetic radiation of determined wavelength;
diluting the mixture by adding a predetermined quantity of water to separate by filtration, the soluble serum proteins from the coagulated caseins suspended in the solution;
acquiring a plurality of right-angle fluorescence spectra of the diluted mixture, the right-angle fluorescence spectra resulting from the excitation of the diluted mixture by at least one electromagnetic radiation with a determined wavelength; and
determining the content of caseins and the content of serum proteins of the liquid foodstuff by applying a multi-path analysis to the frontal fluorescence spectra and to the right-angle fluorescence spectra.

According to different additional characteristics that can be taken together or separately:
a wavelength of at least one electromagnetic radiation is of between 270 nanometres and 340 nanometres, and is preferably equal to 280 nanometres;
the buffer is a buffer of sodium acetate and/or has a pH of between 4.50 and 4.65, preferably equal to 4.55 or to 4.6;
the acquisition of at least one frontal fluorescence spectrum during step c) and/or at least one right-angle fluorescence spectrum during step e) is achieved by a measurement of the diffusion by fluorescence of tryptophan present in the mixture and/or in the diluted mixture;
the liquid foodstuff and the buffer added during step a) have volumes such that the ratio between the liquid foodstuff volume and the buffer volume is of between 1 to 5, and is preferably equal to 3;
the stirring step b) is implemented by means of a vortex; and
the multi-path analysis is a PARAFAC analysis applied to a regression method selected from a multiple linear regression method or a partial least squares regression method.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is a graph illustrating the percentage values of the content of caseins of several milk samples predicted by a multiple linear regression model (on the ordinate) when the steps of the method according to the invention are implemented, represented as a function of the reference content of caseins values (abscissa); and

FIG. 2 is a graph illustrating the percentage values of the content of caseins of several other milk samples predicted by a multiple linear regression model (on the ordinate) when the steps of a method according to the invention are implemented, represented as a function of the reference content of caseins values (abscissa).

### DETAILED DESCRIPTION OF THE INVENTION

A first step of the method according to the invention consists of sampling a volume of a liquid foodstuff to which is added a concentrated buffer.

In an exemplary embodiment according to the invention, a measurement sample of raw milk is prepared and processed as follows. A volume of 7.5 millilitres of milk is mixed with a volume of 2.5 millimetres of a buffer with a pH of 4.55. In a non-limiting manner, the volume of milk mixed with the buffer ranges from 1 millimetre to 10 millimetres, but it can also be selected such that the pH of the mixture of the volume of milk and of the volume of buffer remains as close as possible to that of the initial buffer, at 4.55 in the present case.

Preferably, the buffer is a buffer of sodium acetate. According to other embodiments of the invention, the buffer is a sodium or potassium phosphate buffer. According to other embodiments of the invention, the pH of the buffer is between 4.5 and 4.65.

Preferably, the volume of milk and the volume of buffer are selected so as not to excessively dilute the sample. In particular, a mixture is preferred wherein the ratio of the buffer volume and of the milk volume is included between two tenths and three tenths.

Under the effect of this buffer, the caseins coagulate to form a dense precipitate of coagulable proteins of the foodstuff, this precipitate being suspended in the solution including the soluble serum proteins of the initial milk.

The assembly is then stirred, for example by means of a vortex, to obtain a mixture wherein the precipitate and the solution are homogeneous. Vortex stirring is advantageous with respect to stirring by centrifugation, for example, as it prevents the components of the mixture to stick to the sides of the container wherein the mixture is, in which case it would be more difficult to recover some of them. This mixture is then poured in a tub with sufficient capacity to accommodate it.

After coagulation of the caseins, the caseins contained in the milk tend to diffuse under the effect of light, while the serum proteins are transparent and non-diffusing. Thus, it is possible to establish that caseins are responsible for 99% of the emitted light, while the serum proteins practically do not interfere at all with it, as they are transparent and non-diffusing.

For this purpose, one or more frontal fluorescence spectra emitted by the mixture are acquired, for example, by means of illuminating the sample with a source of monochromatic light of which the wavelength is of between 270 and 300 nanometres, and preferably equal to 280 nanometres. The fluorescence radiations can be recorded by a spectrometer, for example, a spectrometer including a CCD camera. Advantageously, a measurement of the frontal fluorescence makes it possible to determine the full print of the mixture.

The wavelengths of the electromagnetic spectra acquired during the steps of the process according to the invention are preferably between 270 nanometres and 300 nanometres.

In the present invention, tryptophan is used as a marker of the soluble content of serum proteins in an analysed foodstuff. Preferably, the tryptophan present in the supernatant is dosed by fluorescence. The fluorescence of the tryptophan thus makes it possible to determine the concentration in low denatured soluble proteins and is thus strongly correlated to the content of proteins.

Advantageously, the dosage of the tryptophan emitted by the caseins present is optimal for a wavelength of 280 nanometres. The tryptophan dosage could also be performed by the absorption thereof in the ultraviolet.

According to other embodiments, a fluorescence measurement can also be considered by means of at least one wavelength greater than 300 nanometres, for example, 360 nanometres, in view of collecting additional information.

According to an embodiment of the invention, one or more measurements can be taken. In this case, the mixture is stirred again to guarantee the homogeneity of the precipitate and of the solution.

At the end of the application of the method according to the invention, the frontal fluorescence measurement makes it possible to quickly estimate, in about one minute and with an error of less than 3%, the content of caseins of the foodstuff.

A following step of the method according to the invention consists of rendering transparent a sample of the mixture by diluting it with a predetermined quantity of water. Advantageously, this addition of water makes it possible to separate the serum proteins from the precipitated caseins. Moreover, rendering transparent the sample of the mixture with a predetermined quantity of water also makes it possible, according to a corresponding step, to filter it. Thus, a loss of proteins or caseins which would remain stuck in a device is avoided, for example, in a tub containing the sample and the mixture.

A following step of the method according to the invention consists of acquiring a plurality of right-angle fluorescence spectra, also called conventional fluorescence, of the diluted mixture. Right-angle fluorescence spectra result from the excitation of the diluted mixture by at least one electromagnetic radiation of selected wavelength.

According to another embodiment of the invention, the measurement of the frontal fluorescence is measured for a first excitation wavelength of 280 nanometres, for a second excitation wavelength of 340 nanometres. Advantageously, the use of several excitation wavelengths makes it possible to recover a corresponding plurality of emission spectra, which makes it possible to provide additional information as to the content of caseins, the serum content of proteins, or even in other components of the foodstuff. Moreover, the taking of a measurement for an emission wavelength of between 330 and 350 nanometres advantageously makes it possible to obtain a signal of maximum sensitivity.

It is reminded that the scattering (called Rayleigh scattering when the dimension of the particles of the matrix is close to the excitation wavelength, or Mie scattering when it is significantly greater than the wavelength) corresponds to an elastic interaction (without energy loss) between the light at the considered wavelength and the matrix. This interaction considers the structure of the matrix, and in particular, the size and the concentration of the particles with a dimension close to the wavelength.

According to an embodiment of the method according to the invention, a scattering filtration step is provided, which advantageously avoids polluting the right-angle fluorescence measurement.

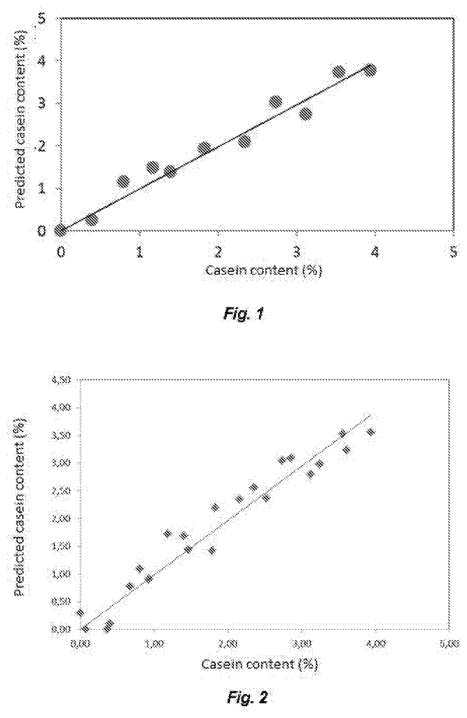

FIG. 1 represents a first application of the invention with several milk samples.

After having prepared a volume of each sample to obtain a homogeneous mixture of a precipitate of caseins suspended in a solution that includes soluble proteins, the frontal fluorescence of this mixture was measured by means of two excitation wavelengths, one being equal to 280 nanometres and the other being equal to 340 nanometres.

The spectral data collected for each sample have then been organised in a large dimension matrix, called “excitation-emission matrix” (EEM), one dimension of which represents the emission wavelengths, and the other dimension represents the emission wavelengths. The EEM thus obtained was then analysed by a mathematical analysis method, which makes it possible to extract the information corresponding to the bilinear profiles of fluorescence and to the relative intensities thereof. Then, a multiple linear regression method was applied to predict the value of the content of caseins for each sample of the foodstuff.

In view of implementing the invention, a calibration phase can be conducted beforehand. Before determining the content of caseins of a given sample by means of the method according to the invention, this calibration can be implemented by means of a mathematical analysis. As a first example, a principal component analysis (PCA) or a partial least squares analysis (PLS) is implemented when a single wavelength is used. As a second example, a PARAFAC-type analysis or any other multi-path decomposition method is implemented if more than one wavelength is used.

With reference to FIG. 1, each sample includes a content of caseins that increases progressively. In this first application, different volumes of a highly concentrated casein powder were added to a milk sample, while keeping constant the soluble content of proteins.

In FIG. 1, a simple model by multiple linear regression, or by partial least squares regression (PLS) is applicable following the decomposition of the excitation-emission matrixes to determine the content of caseins. A PARAFAC-type model is applicable if the sample is illuminated by several light radiations or by a continuous light source. The predicted value thus obtained is shown on the Y axis as a function of the casein reference values, expressed in grams of casein per 100 grams of sample, in order to construct the calibration. The results obtained show that the calibration as indicated, the calibration shows that a square root of the calibration equal to 0.283%, and a square root of the crossed validation equal to 0.320% have been obtained, which corresponds to a relative error of 10%.

These results illustrate the excellent correlation existing between the content of caseins obtained with the method according to the invention applied to various milk samples, and the reference content values.

A second application of the invention to several milk samples was also tested, each of these samples including a content in soluble proteins that increases progressively. In this second application, different volumes of a soluble serum protein powder were added to a milk sample, while keeping constant the soluble content of proteins.

For all the samples of this application, the average concentration of caseins was quantified as being of 0.25% with a standard deviation of 3%, which is compatible with the reproducibility error of the measurements. The soluble protein fraction has been measured and has confirmed reliable results, consistent with those of patent application FR 2 752 941.

FIG. 2 represents a third application of the invention to several samples, each sample including a volume of micro-filtered milk retentate increasing progressively, so as to have different samples of which the content of caseins and of soluble serum proteins is highly variable.

After homogenising each sample, the corresponding frontal fluorescence measurements have been taken by illuminating each of them with a first wavelength of 280 nanometres and a second wavelength of 340 nanometres.

By using a PARAFAC decomposition method followed by a multiple linear regression method, the results represented in FIG. 2 show that a reliable calibration of the predicted content of caseins is obtained for each sample (on the ordinate), with respect to the reference content of caseins values expressed in grams of casein per 100 grams of sample.

On the basis of a liner regression, of which the equation for the curve is defined by y(x)−0.984 x, the calibration results show that a square root of the calibration error equal to 0.34% is obtained, which highlights the great variability of the samples in this third application. Moreover, obtaining a square root of the crossed validation error equal to 0.56% indicates that a strong calibration requires, ideally, a larger number of samples. However, because of the speed of the method, several hundreds of samples can be measured per day. Of note, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes”, and/or “including,” when used in this specification, specify the presence of stated features, integers, steps, operations, elements, and/or components, but do not preclude the presence or addition of one or more other features, integers, steps, operations, elements, components, and/or groups thereof.

As well, the corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

What is claimed is:

1. A method for determining a content of caseins and a content of serum proteins in a liquid foodstuff such as raw or low pasteurized milk, comprising:
a) forming a solution of said liquid foodstuff by adding a buffer with a predetermined pH, said solution comprising said serum proteins and said caseins, the caseins being in a coagulated form suspended in the solution and the serum proteins being solubilized in the solution;
b) stirring the solution to form a homogeneous mixture of the serum proteins and caseins;
c) acquiring a plurality of frontal fluorescence spectra of said mixture, said frontal fluorescence spectra resulting from the excitation of said homogeneous mixture by at least one electromagnetic radiation with a determined wavelength;
d) diluting the mixture by adding a predetermined quantity of water to separate by filtration the soluble serum proteins from the coagulated caseins suspended in the solution.
e) acquiring a plurality of right-angle fluorescence spectra of said diluted mixture, said right-angle fluorescence spectra resulting from the excitation of said diluted mixture by at least one electromagnetic radiation with a determined wavelength; and
f) determining the content of caseins and the content of serum proteins of the liquid foodstuff by applying a multi-path analysis to the frontal fluorescence spectra and to the right-angle fluorescence spectra.

2. The method according to claim 1, wherein a wavelength of at least one electromagnetic radiation is comprised between 270 nanometres and 340 nanometres, and is preferably equal to 280 nanometres.

3. The method according to claim 1, wherein said buffer is a buffer of sodium acetate and/or has a pH of between 4.50 and 4.65, preferably equal to 4.55 or 4.6.

4. The method according to claim 1, wherein the acquisition of at least one frontal fluorescence spectrum during step c) and/or at least one right-angle fluorescence spectrum during step e) is achieved by a measurement of the tryptophan present in the mixture and/or in the diluted mixture.

5. The method according to claim 1, wherein the liquid foodstuff and the buffer added during step a) have volumes such that the ratio between the liquid foodstuff volume and the buffer volume is comprised between 1 and 5, and is preferably equal to 3.

6. The method according to claim 1, wherein the stirring step b) is implemented by means of a vortex.

7. The method according to claim 1, wherein said multi-path analysis is a PARAFAC analysis applied to a regression method selected from a multiple linear regression method or a partial least squares regression method.
